(19) United States
(12) Patent Application Publication
(10) Pub. No.: US 2012/0291523 A1
(43) Pub. Date: Nov. 22, 2012

(54) KIT FOR ASSESSING THE FRAGRANCE INTENSITY OF A FABRIC CARE PRODUCT

(76) Inventors: Citra (Wijaya) Ng, Beijing (CN); Shaokun Lin, Beijing (CN); Joseph (Tan) Chio, Beijing (CN); Ricardo Leal Macias, Beijing (CN)

(21) Appl. No.: 13/472,548

(22) Filed: May 16, 2012

(30) Foreign Application Priority Data
May 18, 2011 (CN) PCT/CN2011/074265

(51) Int. Cl. G01N 33/00

(52) U.S. Cl. 73/23.34

(57) ABSTRACT

The present invention relates to a kit for assessing the fragrance intensity of a fabric care product and a method for assessing the fragrance intensity imparted by a fabric care product comprising perfume microcapsules. The kit works instantly and ensures steady assessing conditions without requiring complicated instruments or professional operators.

### FIELD OF THE INVENTION

The present invention relates to a kit for assessing the fragrance intensity of a fabric care product. The present invention also relates to a method for assessing the fragrance intensity imparted by a fabric care product comprising perfume microcapsules.

### BACKGROUND OF THE INVENTION

Perfume microcapsules have been used in fabric care products for several years. Perfume microcapsules comprise a wall material and a core material of perfume that is encapsulated within the wall material. The perfume is substantially not being released from the microcapsules until the wall material ruptures because of a mechanical stress (e.g., friction). Perfume microcapsules are added into fabric care products to provide improved delivery efficiency of perfume. Specifically, perfume capsules deposit onto fabrics during a wash or rinse cycle. The perfume within perfume microcapsules is protected from volatilization to the surrounding air space for a prolonged duration of time. Such, perfume capsules, when deposited on fabrics, exhibit a burst of perfume upon rupturing.

Methods for assessing the fragrance intensity of perfume microcapsules or fabric care products comprising such perfume microcapsules have been disclosed in the art. However, there are some limitations to the convenience and efficiency of the existing methods. In particular, most of the methods require complicated instruments, professional operators and time consuming procedures. In addition, there are relatively simple methods. However, these simple methods do not ensure steady assessing conditions, and thus are not reliable.

It is thus an objective of the present invention to provide a kit for assessing the fragrance intensity of a fabric care product comprising perfume microcapsules that provides a convenient and efficient assessment. Specifically, it is an objective of the present invention to provide a kit for assessing the fragrance intensity of a fabric care product comprising perfume microcapsules that works instantly without requiring complicated instruments or professional operators.

It has been found that the above objective can be met by the kit of the present invention.

It is an advantage of the present invention to provide a kit for demonstrating the benefits of perfume microcapsules, i.e., without or small perfume leakage until being activated and causing a burst of perfume upon rupturing.

It is a further advantage of the present invention to provide a kit that allows users to have a delightful scent experience and an impression about perfume microcapsules. Specifically, it is an advantage of the present invention to provide a kit that gives users a good idea of how their fabrics will smell when they use the fabric care products comprising perfume microcapsules.

### SUMMARY OF THE INVENTION

The present invention relates to a kit 1 for assessing the fragrance intensity of a fabric care product, wherein the kit 1 comprises:

a) a container 2 comprising an opening 3;

b) an array 7 of sheets 6 disposed within the container 2, comprising at least two sheets 6, wherein at least 33% of the surface area of each sheet 6 in the array 7 is in contact with one or more sheets 6 forming part of the array 7; and

c) the fabric care product comprising perfume microcapsules wherein the fabric care product is applied onto the sheets 6;

wherein the sheets 6 have an ability to release a baseline fragrance intensity and are individually removed from the container 2 through the opening 3 by a user and wherein friction caused by such removal causes a fragrance intensity from the sheets 6 which is at least about 5 grades greater than the baseline fragrance intensity.

The present invention further relates to a method for assessing the fragrance intensity imparted by a fabric care product comprising perfume microcapsules, wherein the method comprises steps of a) providing the kit 1 and b) removing the sheets 6 from the container 2 through the opening 3.

### BRIEF DESCRIPTION OF THE DRAWINGS

FIG. 1 is an isometric view of a container forming part of the kit according to one embodiment of the present invention.

FIG. 2 is a top view of an opening of a container forming part of the kit according to one embodiment of the present invention.

FIG. 3 is a top view of an opening of a container forming part of the kit according to an alternative embodiment of the present invention.

FIG. 4 is a top view of an opening of a container forming part of the kit according to another alternative embodiment of the present invention.

FIG. 5 is an isometric view of an array of sheets of a kit according to one embodiment of the present invention.

FIG. 6 is a side view of the array of sheets in FIG. 5.

FIG. 7 is a side view of an array of sheets of a kit according to another embodiment of the present invention.

FIG. 8 is an isometric view of a kit according to an alternative embodiment of the present invention.

### DETAILED DESCRIPTION OF THE INVENTION

### Definitions

The term “fabric care product” as used herein includes, unless otherwise indicated, fabric softening products, fabric enhancer products, fabric freshening products; granular or powder-form all-purpose or “heavy-duty” washing agents, especially cleaning detergents; liquid, gel or paste-form all-purpose washing agents, especially the so-called heavy-duty liquid types, liquid fine-fabric detergents; including the various tablet, granular, liquid and rinse-aids, sprays and mists types for fabrics for household and institutional use; as well as cleaning auxiliaries such as bleach additives or pre-treat types, substrate-laden products such as dryer added sheets, dry and wetted wipes and pads, and nonwoven substrates.

As used herein, the terms “perfume”, “fragrance” and “scent” are synonymous.

As used here, the term “concentration” refers to the amount of a specified constituent in a unit amount of a mixture.

As used herein, the articles including “a” and “an” when used in a claim, are understood to mean one or more of what is claimed or described.

As used herein, the terms “include”, “includes” and “including” are meant to be non-limiting.

All percentages and ratios are calculated by weight unless otherwise indicated. All percentages and ratios are calculated based on the total composition unless otherwise indicated.

Unless otherwise noted, all component or composition levels are in reference to the active level of that component or composition, and are exclusive of impurities, for example, residual solvents or by-products, which may be present in commercially available sources.

### Container

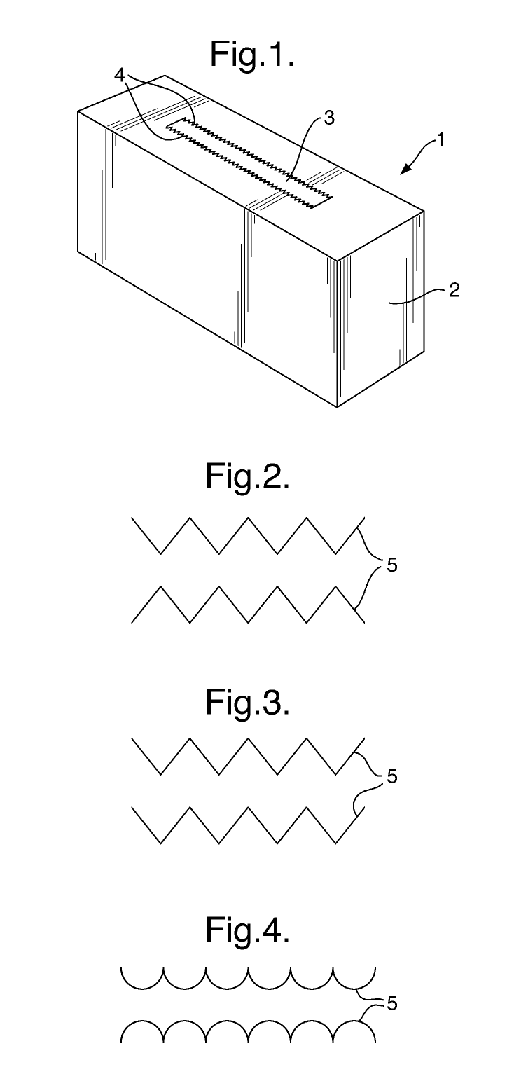

As shown in FIGS. 1 and 8, the kit 1 of the present invention comprises a container 2.

The container 2 herein can be of any shape known to the person skilled in the art, including polyhedral, sphere, and cylinder.

In a preferred embodiment, the container 2 is a cube or cuboid. In an even more preferred embodiment, the container 2 is a cuboid having a length of from 5 cm to 100 cm, preferably of from 10 cm to 30 cm, a depth of from 3 cm to 100 cm, preferably of from 5 cm to 25 cm, and a height of from 5 cm to 100 cm, preferably of from 10 cm to 20 cm.

In another preferred embodiment of the present invention, the container 2 is an elliptic cylinder or a right circle cylinder. In a more preferred embodiment, the container 2 is a right circle cylinder with a radius of from 2 cm to 50 cm and a height of from 5 cm to 100 cm.

In another embodiment, the container 2 is a sphere with a radius of from 2 cm to 50 cm.

The container 2 herein can be made of any material known to the person skilled in the art. Preferably, the container 2 is made of materials selected from the group consisting of plastic, paper, cartoon, and metal plate, and a combination thereof.

The container 2 herein may have pictures, product logos, or text applied to its walls. Preferably, such pictures, product logos, or text is applied, preferably printed, onto the outward facing side of at least parts of the container's walls. For example, the container 2 is printed with the logo or text related to the fabric care product, or the artwork that is the same or similar with that printed on the packaging of the fabric care product. The container 2 may also be of a specific color, such as the color of the packaging of the fabric care product.

In a highly preferred embodiment, the container 2 herein is a cuboid shaped box printed with the logo or text used for the fabric care product and the artwork that is similar with that printed on the packaging of the fabric care product.

### Opening

As shown in FIGS. 1 and 8, the container 2 comprises an opening 3. The opening 3 may be located in any part of the container 2. The opening 3 may be on one side of the container 2 or spanning two or more sides of the container 2. Preferably, in normal operation of the container 2, the opening 3 is located on the upper half of the container 2, preferably on the top part of the container 2.

The opening 3 herein can be of any shape, such as triangular, rectangular, pentagonal, hexagonal, oval, or round. Preferably, the shape of the opening 3 is rectangular, oval, or round.

In a preferred embodiment, the opening 3 is a rectangle having two parallel long edges and two parallel short edges. Even more preferably, the length of the short edges is from 0.05 mm to 40 mm, preferably from 0.1 mm to 5 mm, more preferably from 0.1 mm to 1.5 mm, and the length of the long edges is from 5 mm to 500 mm, preferably from 10 mm to 200 mm, more preferably from 15 mm to 150 mm.

In another preferred embodiment, the shape of the opening 3 is oval. More preferably, the long diameter of the oval shaped opening 3 is from 1.5 mm to 200 mm, preferably from 5 mm to 200 mm, and the short diameter is from 0.05 mm to 60 mm, preferably from 1.5 mm to 60 mm.

In another preferred embodiment, the shape of the opening 3 is round. More preferably, the radius of the round shaped opening 3 is from 1.5 mm to 100 mm, preferably from 5 mm to 100 mm.

The opening 3 herein may have one or more edges 4 with protrusions 5 extending from the edge portion of the opening 3 into the opening portion. The protrusions 5 may be the same or different shape and/or dimensions along the edges 4 or may even be absent in certain parts of the opening 3. The shape of the protrusions 5 is preferably selected from the group consisting of triangular, pentagonal, hexagonal, oval, and round, and a combination thereof. More preferably, as shown in FIGS. 2 and 3, the shape of the protrusions 5 is triangular. Specifically, in FIG. 2, the triangular protrusions 5 on two edges 4 are opposing, whereas those in FIG. 3 are complementary. In another embodiment, as shown in FIG. 4, the shape of the protrusions 5 is round.

Preferably, protrusion density along any one of the edges 4 (if any protrusions are present) is from 0.5 to 200, preferably from 0.25 to 37.5, more preferably from 0.125 to 25 protrusions per 1 cm length. The protrusion density may be the same or different on the different edge portions of a given container. The term “protrusion density” as used herein means the amount of the protrusions along a unit length of the edge.

More preferably, protrusion depth along any one of the edges 4 (if any protrusions are present) is from 0.05 mm to 20 mm, preferably from 0.1 mm to 5 mm, more preferably from 0.1 mm to 1.5 mm. The protrusion depth may be the same or different on the different edge portions of a given container. The term “protrusion depth” as used herein means the vertical distance between the baseline of the edges and the furthest point of the extended protrusion into the opening portion.

The container 2 herein may further comprise a lid to cover the opening 3, and the lid has the same or different shape with the opening 3. Preferably, the lid has the same shape with the opening 3. More preferably, the lid is slightly larger than the opening 3.

In another embodiment, the material forming the edge portion of the opening 3 is the same or different from the material of the container 2. The material forming the edge portion of the opening 3 may be an integral part of the material of the container 2 or a separate piece that is mounted to the material of the container 2. The material forming the edge portion of the opening 3 is preferably selected from the group consisting of plastic, paper, cartoon, and metal plate, and a combination thereof in preferably two or more layers.

In a highly preferred embodiment, as shown in FIG. 1, the opening 3 is located on one side of the container 2, is a rectangle having two parallel long edges and two parallel short edges, wherein the length of the short edges is 0.1 mm and the length of the long edges is 150 mm, and has two edges 4 with protrusions 5 extending from the edge portion of the opening 3 into the opening portion, wherein the protrusion density is 12.5 protrusions per 1 cm length and the protrusion density is 1 mm.

### Sheets

The kit 1 of the present invention further comprises an array 7 of sheets 6 disposed within the container 2, wherein the array 7 of sheets 6 comprises at least two sheets 6 and at least 33% of the surface area of each sheet 6 in the array 7 is in contact with one or more sheets 6 forming part of the array 7.

Preferably, in the array 7 of sheets 6, from 33% to 95%, preferably from 50% to 85%, of the surface area of each sheet 6 in the array 7 is in contact with one or more sheets 6 forming part of the array 7.

The array 7 of sheets 6 herein can comprise from two to any suitable number of sheets known to the person skilled in the art. Preferably, the array 7 of sheets 6 comprises from 2 to 1000 sheets, preferably from 2 to 500 sheets, more preferably from 2 to 200 sheets, even more preferably from 2 to 50 sheets.

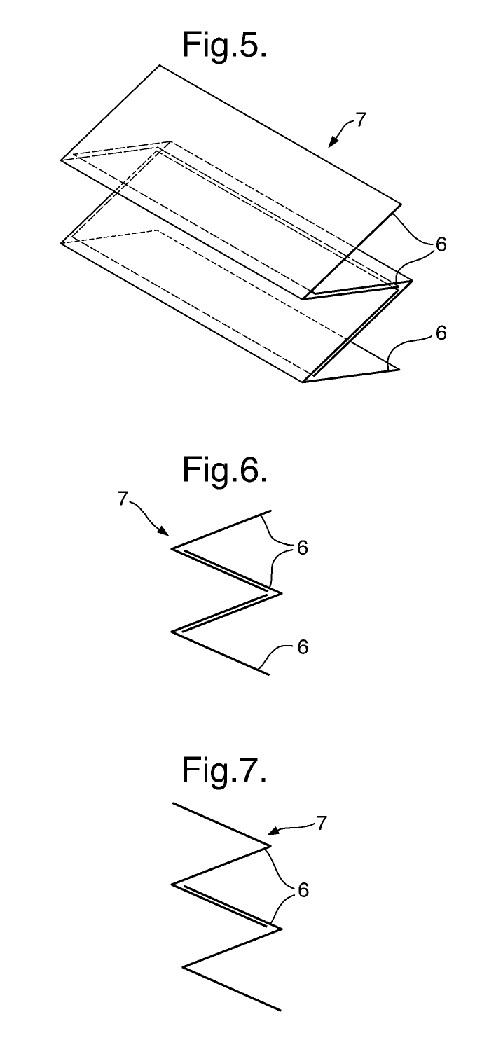

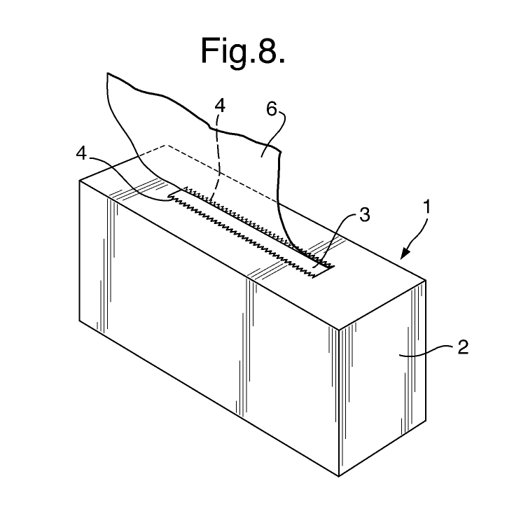

The array 7 of sheets 6 herein may be of any arrangement, such as a roll of sheets 6, a stack of sheets 6, or random assembly of sheets 6. In a preferred embodiment, the array 7 of sheets 6 is a roll of sheets 6. As shown in FIG. 7, in another preferred embodiment, the array 7 of sheets 6 is a stack of sheets 6. Most preferably, as shown in FIGS. 5 and 6, each sheet 6 overlaps with the neighboring sheet 6 over at least 50% of the surface area of one side of the sheet 6.

The array 7 of sheets 6 herein can be disposed into the container 2 directly, or wrapped in a bag first and then put into the container 2. The bag for wrapping the array 7 of sheets 6 can be made of any suitable material known to the person skilled in the art. Preferably, the bag is made of materials selected from the group consisting of plastic, paper, cartoon, and metal plate, and a combination thereof. More preferably, the bag is made of plastic.

In one embodiment, especially when the array 7 of sheets 6 is prepared in a first site and disposed into the container 2 in a second site, the array 7 of sheets 6 is wrapped in a bag and shipped to the second site.

In a preferred embodiment, in the second site, the array 7 of sheets 6 is unwrapped by removing the bag and disposed into the container 2. The bag is preferably a plastic bag or aluminum foil.

In another preferred embodiment, in the second site, the array 7 of sheets 6 in the bag is disposed into the container 2 without removing the bag. Preferably, the bag is a plastic bag. More preferably, the bag has a perforation line on one of its side or spanning two or more of its sides. Even more preferably, the perforation line is in line with the position of the opening 3 on the container 2. Such perforation line enables users to pull out the sheet 6 through the perforation line prior to the sheet 6 going through the opening 3 out of the container 2.

The sheets 6 herein can be made of any suitable material or a combination thereof known to the person skilled in the art. Preferably, the material of the sheets 6 is selected from the group consisting of cotton, poly cotton, nylon, paper, and nonwoven, and a combination thereof. More preferably, the material of the sheets 6 includes cotton. Most preferably, the material of the sheets 6 is 100% cotton.

The sheets 6 herein can be of any shape, such as triangular, rectangular, pentagonal, hexagonal, oval, or round. The sheet 6 also can be of any size known to the person skilled in the art. Preferably, the sheet 6 has a size matching with the dimension of the container 2 and the opening 3, thus easy to be removed out of the container 2 through the opening 3. In a preferred embodiment, each sheet is a rectangle with a length of from 3 cm to 100 cm and a width of from 2 cm to 100 cm.

In a preferred embodiment, the thickness of each sheet 6 is from 0.01 mm to 3 mm, preferably from 0.05 mm to 1.0 mm, more preferably from 0.10 mm to 0.60 mm. In yet another embodiment, the basis weight of each sheet 6 is from 0.125 grams to 7.5 grams per 100 square centimeters.

The sheets 6 herein may have pictures, product logos, or text. Preferably, such pictures, product logos, or text is applied, preferably printed, onto the sheets 6. For example, the sheets 6 are printed with the logo or text related to the fabric care product, or the artwork that is the same or similar with that printed on the packaging of the fabric care product. Preferably, the sheets 6 are printed with a text “please rub me”. The sheets 6 may also be of a specific color, such as the color of the packaging of the fabric care product.

In a highly preferred embodiment, the array 7 of sheets 6 is a stack of sheets, wherein at least 50% of the surface area of each sheet 6 in the array 7 is in contact with one or more sheets 6 forming part of the array 7, as shown in FIGS. 5, 6, and 7, and each sheet 6 is printed with text, such as “please rub me”.

### Fabric Care Product

The kit 1 of the present invention comprises a fabric care product comprising perfume microcapsules.

The fabric care product herein may comprise a laundry detergent composition. The laundry detergent composition may be in solid or liquid form, including a gel form. Preferably, the composition is liquid in form and comprises heavy duty liquid compositions. The composition comprises a surfactant in an amount sufficient to provide desired cleaning properties. Preferably, the laundry detergent composition comprises, by weight, from 5% to 90% of a surfactant, and more preferably from 5% to 70% of a surfactant, and even more preferably from 5% to 40% of a surfactant. The surfactant may preferably comprise anionic, nonionic, cationic, zwitterionic and/or amphoteric surfactants. In a more preferred embodiment, the detergent composition comprises anionic surfactant, nonionic surfactant, or mixtures thereof.

The fabric care product herein may comprise a fabric softening active (“FSA”). Suitable fabric softening actives, include, but are not limited to, materials selected from the group consisting of quats, amines, fatty esters, sucrose esters, silicones, dispersible polyolefins, clays, polysaccharides, fatty oils, polymer latexes, and mixtures thereof. Typical minimum levels of incorporation of the FSA in the present fabric care products are at least 1%, alternatively at least 2%, alternatively at least 3%, alternatively at least 5%, alternatively at least 10%, and alternatively at least 12%, by weight of the fabric care composition. The fabric care composition may typically comprise maximum levels of FSA of less than 90%, alternatively less than 40%, alternatively less than 30%, alternatively less than 20%, by weight of the composition.

In a preferred embodiment herein, the fabric care product comprises a perfume ingredient in addition to the perfume present in the perfume microcapsules present in the fabric care product.

### Perfume Microcapsules

Encapsulation of perfume or other materials in small capsules (i.e., microcapsules), typically having a diameter less than 300 microns, is generally well known. Typically, these microcapsules comprise a spherical hollow shell of water insoluble material, typically polymer material, within which the active material, such as perfume, is contained. Microcapsules are described in the following references: US 2003/215417 A1; US 2003/216488 A1; US 2003/158344 A1; US 2003/165692 A1; US 2004/071742 A1; US 2004/071746 A1; US 2004/072719 A1; US 2004/072720 A1; EP 1,393,706 A1; US 2003/203829 A1; US 2003/195133 A1; US 2004/087477 A1; US 2004/0106536 A1; U.S. Pat. No. 6,645,479; U.S. Pat. No. 6,200,949; U.S. Pat. No. 4,882,220; U.S. Pat. No. 4,917,920; U.S. Pat. No. 4,514,461; US RE 32,713; U.S. Pat. No. 4,234,627. Microcapsules may be prepared using a range of conventional methods known to those skilled in the art for making shell capsules, such as Interfacial polymerization, and polycondensation. See e.g., U.S. Pat. No. 3,516,941, U.S. Pat. No. 4,520,142, U.S. Pat. No. 4,528,226, U.S. Pat. No. 4,681,806, U.S. Pat. No. 4,145,184; GB 2,073,132; WO 99/17871; and MICROENCAPSULATION: Methods and Industrial Applications Edited by Benita and Simon (Marcel Dekker, Inc. 1996). It is recognized, however, that many variations with regard to materials and process steps are possible. Non-limiting examples of materials suitable for making shell of the microcapsule include urea-formaldehyde, melamine-formaldehyde, phenol-formaldehyde, gelatin, polyurethane, polyamides.

In one embodiment of the present invention, the shell of the microcapsules comprises an aminoplast resin. A method for forming such shell capsules includes polycondensation. Aminoplast resins are the reaction products of one or more amines with one or more aldehydes, typically formaldehyde. Non-limiting examples of suitable amines include urea, thiourea, melamine and its derivates, benzoguanamine and acetoguanamine and combinations of amines. Suitable cross-linking agents (e.g., toluene diisocyanate, divinyl benzene, butanediol diacrylate etc.) may also be used and secondary wall polymers may also be used as appropriate, e.g. anhydrides and their derivatives, particularly polymers and co-polymers of maleic anhydride as disclosed in WO 02/074430. In another embodiment, the shell of the microcapsules comprises urea-formaldehyde; melamine-formaldehyde; or combinations thereof.

The perfume microcapsules of the present invention should be friable in nature. Friability refers to the propensity of the microcapsules to rupture or break open when subjected to direct external pressures or shear forces. For purposes of the present invention, the microcapsules utilized are “friable” if, while attached to the sheets 6 treated therewith, they can be ruptured by the forces encountered and thereby release the perfume when the capsule-containing sheets 6 are manipulated by causing friction. Microcapsules of the type utilized herein have friability characteristics such that the ordinary manipulation which occurs when the treated sheets 6 are removed from the container 2 through the opening 3 is sufficient for attached microcapsules to impart a noticeable odor. The perfume within the microcapsules of the present invention surprisingly maximizes the effect of the microcapsules bursting by providing a perfume that “blooms” upon the microcapsules rupturing.

In one embodiment, the shell capsules typically have a mean diameter in the range 1 micrometer to 100 micrometers, alternatively from 5 micrometers to 80 microns, alternatively from 10 micrometers to 75 micrometers, and alternatively between 15 micrometers to 50 micrometers. The particle size distribution can be narrow, broad, or multimodal.

In another embodiment, microcapsules vary in size having a maximum diameter between 5 microns and 300 microns, alternatively between 10 microns and 200 microns. As the capsule particle size approaches 300 microns, e.g. 250 microns, a reduction in the number of capsules entrained in the sheet 6 may be observed.

In another embodiment, the capsules utilized in the present invention generally have an average shell thickness ranging from 0.1 micron to 50 microns, alternatively from 1 micron to 10 microns.

In another embodiment, the perfume microcapsules comprise a loading/complexation level of from 50% to 90%, alternatively from 60% to 80%, alternatively from 65% to 75%, by weight of a blooming perfume composition. This loading/complexation property of the perfume microcapsules of the present invention is advantageous versus other technologies such as beta-cyclodextrin. The advantages may include, but are not limited to, one or more of the following: (i) the ability to use a reduced total perfume level, e.g., in neat perfume (direct add); in perfume microcapsules; or combinations thereof; (ii) avoiding cost in processing and lost material through processing; (iii) delivering a high level of perfume while not affecting process product disposition or process parameters; and (iv) delivering a high level of perfume to fabric while avoiding a high level of neat product odor, which can be a consumer negative.

Suppliers of microcapsules may include International Flavors & Fragrances (IFF), Reed Pacific, and Appleton. An example of a suitable microcapsule for purposes of the present invention includes Perfume Microcapsules from Appleton. Other examples may include WIZARD from Reed Pacific, and EVERLAST from IFF. For a preferred embodiment, the shell is formed by cross-linking aldehydes and amine functionalities. In one embodiment, the encapsulated blooming perfume may, in one embodiment, comprise from 3 to 300 different perfume ingredients, preferably with minimal modifiers which include viscosity or hydrophobicity modifiers. Typical viscosity modifiers include, but not limited to, silicone oil, gums, and waxes. Typical hydrophobic modifiers include, but not limited to, isopropyl myristate, mineral oil, dipropylenemethyl ether (DPM). Such modifiers may be used at less than 50%, alternatively less than 40%, alternatively less than 30%, alternatively less than 20%, alternatively less than 10%, alternatively less than 5%, alternatively less than 1%, alternatively 0%, alternatively at least 0.1% but not greater than 50%, by weight of total perfume composition. Without wishing to be bound by theory, the overuse of modifiers reduces the efficiency of the scent experience imparted by the perfume microcapsules of the present invention.

In one embodiment of the present invention, the perfume microcapsules in said fabric care product for preparing said sheets 6 is able to deliver a solution with a concentration of perfume microcapsules from 0.1 ppm to 200 ppm, preferably from 0.2 ppm to 100 ppm, more preferably from 0.3 ppm to 50 ppm.

The present invention is based, in part, upon the surprising discovery that the blooming perfume composition of the present invention maximizes the opportunity for a user to go through a unique scent experience, i.e., a burst of scent while using the kit 1 of the present invention. In one embodiment, the perfume microcapsule encapsulates a blooming perfume composition, wherein the blooming perfume composition, in the absence of water, comprises from 5% to 95%, alternatively from 20% to 90%; alternatively from 25% to 85%, by the total weight of the perfume microcapsule and the encapsulated perfume composition, also in absence of water.

The term “blooming perfume composition” as used herein means a perfume composition that comprises at least 25%, alternatively at least 35%, alternatively at least 45%, alternatively at least 55%, alternatively at least 65%, by weight of the perfume composition, of blooming perfume ingredients, wherein the blooming perfume ingredients are those having a boiling point (B.P.) equal to or lower than 250° C., more preferably equal to or lower than 250° C., wherein the B.P. is measured at the normal standard pressure.

The boiling points of many perfume ingredients are given in, e.g., “Perfume and Flavor Chemicals (Aroma Chemicals),” S. Arctander, published by the author, 1969. Other boiling point values can be obtained from different chemistry handbooks and databases, such as the Beilstein Handbook, Lange's Handbook of Chemistry, and the CRC Handbook of Chemistry and Physics. When a boiling point is given only at a different pressure, usually at a pressure lower than the standard pressure (760 mm Hg), the boiling point at standard pressure can be approximately estimated by using boiling point-pressure monographs, such as those given in “The Chemist's Companion,” A. J. Gordon and R. A. Ford, John Wiley & Sons Publishers, 1972, pp. 30-36. When applicable, the boiling point values can also be calculated by computer programs, based on molecular structural data, such as those described in “Computer-Assisted Prediction of Normal Boiling Points of Pyrans and Pyrroles,” D. T. Stanton et al, J. Chem. Inf. Comput. Sci., 32 (1992), pp. 306-316, “Computer-Assisted Prediction of Normal Boiling Points of Furans,” Tetrahydrofurans, and Thiophenes,” D. T. Stanton et al, J. Chem. Inf. Comput. Sci., 31 (1992), pp. 301-310, and references cited therein, and “Predicting Physical Properties from Molecular Structure,” R. Murugan et al, Chemtech, June 1994, pp. 17-23.

Non-limiting examples of blooming perfume ingredients that are useful in the articles of the present invention are given in U.S. Pat. Pub. No. 2005/0192207 A1, published Sep. 1, 2005, 29-31.

In one embodiment, the blooming perfume compositions of the present invention comprises at least 3 different blooming perfume ingredients, preferably at least 4 different blooming perfume ingredients, more preferably at least 5 different blooming perfume ingredients, and even more preferably at least 6 different blooming perfume ingredients.

In the perfume art, some materials having no odor or very faint odor are used as diluents or extenders. Non-limiting examples of these materials are dipropylene glycol, diethyl phthalate, triethyl citrate, isopropyl myristate, and benzyl benzoate. These materials are used for, e.g., diluting and stabilizing some other perfume ingredients. For purposes of this invention, these materials are not counted as a “blooming perfume ingredient.”

In one embodiment, the perfume ingredient that comprises the blooming perfume composition is chosen from at least of the following: blooming perfume ingredient is chosen from the group consisting of allo-ocimene, allyl caproate, allyl heptoate, amyl propionate, anethol, anisic aldehyde, anisole, benzaldehyde, benzyl acetate, benzyl acetone, benzyl alcohol, benzyl butyrate, benzyl formate, benzyl iso valerate, benzyl propionate, beta gamma hexenol, camphene, camphor, carvacrol, laevo-carveol, d-carvone, laevo-carvone, cinnamyl formate, citral (neral), citronellol, citronellyl acetate, citronellyl isobutyrate, citronellyl nitrile, citronellyl propionate, cuminic alcohol, cuminic aldehyde, Cyclal C, cyclohexyl ethyl acetate, decyl aldehyde, dihydro myrcenol, dimethyl benzyl carbinol, dimethyl benzyl carbinyl acetate, dimethyl octanol, diphenyl oxide, ethyl acetate, ethyl aceto acetate, ethyl amyl ketone, ethyl benzoate, ethyl butyrate, ethyl hexyl ketone, ethyl phenyl acetate, eucalyptol, eugenol, fenchyl acetate, fenchyl alcohol, flor acetate (tricyclo decenyl acetate), frutene (tricyclo decenyl propionate), gamma methyl ionone, gamma-n-methyl ionone, gamma-nonalactone, geraniol, geranyl acetate, geranyl formate, geranyl isobutyrate, geranyl nitrile, hexenol, hexenyl acetate, cis-3-hexenyl acetate, hexenyl isobutyrate, cis-3-hexenyl tiglate, hexyl acetate, hexyl formate, hexyl neopentanoate, hexyl tiglate, hydratropic alcohol, hydroxycitronellal, indole, isoamyl alcohol, alpha-ionone, beta-ionone, gamma-ionone, alpha-irone, isobornyl acetate, isobutyl benzoate, isobutyl quinoline, isomenthol, isomenthone, isononyl acetate, isononyl alcohol, para-isopropyl phenylacetaldehyde, isopulegol, isopulegyl acetate, isoquinoline, cis-jasmone, lauric aldehyde (dodecanal), Ligustral, d-limonene, linalool, linalool oxide, linalyl acetate, linalyl formate, menthone, menthyl acetate, methyl acetophenone, methyl amyl ketone, methyl anthranilate, methyl benzoate, methyl benzyl acetate, methyl chavicol, methyl eugenol, methyl heptenone, methyl heptine carbonate, methyl heptyl ketone, methyl hexyl ketone, alpha-iso “gamma” methyl ionone, methyl nonyl acetaldehyde, methyl octyl acetaldehyde, methyl phenyl carbinyl acetate, methyl salicylate, myrcene, neral, nerol, neral acetate, nonyl acetate, nonyl aldehyde, octalactone, octyl alcohol (octanol-2), octyl aldehyde, orange terpenes (d-limonene), para-cresol, para-cresyl methyl ether, para-cymene, para-methyl acetophenone, phenoxy ethanol, phenyl acetaldehyde, phenyl ethyl acetate, phenyl ethyl alcohol, phenyl ethyl dimethyl carbinol, alpha-pinene, beta-pinene, prenyl acetate, propyl butyrate, pulegone, rose oxide, safrole, alpha-terpinene, gamma-terpinene, 4-terpinenol, alpha-terpineol, terpinolene, terpinyl acetate, tetrahydro linalool, tetrahydro myrcenol, tonalid, undecenal, veratrol, verdox, vertenex, viridine, and combinations thereof.

In one embodiment, substantive perfume ingredients, which can be used as part of blooming perfume compositions in articles of the present invention, are those having a B.P. higher than 250° C. Non-limiting examples of such perfume ingredients include those described in U.S. Pat. Pub. No. 2005/0192207 A1, published Sep. 1, 2005.

Another aspect of the invention provides for the article to comprise an optional perfume component comprising at least one of the following: (a) a moisture-activated perfume microcapsule comprising a perfume carrier and an encapsulated perfume composition; (b) a pro-perfume; (c) a low odor detection threshold perfume ingredients; (d) neat perfume; and (e) mixtures thereof. In one embodiment, the article is free or substantially free of any one or more of the aforementioned perfume components.

The kit 1 of the present invention provides a convenient and efficient assessment of the fragrance intensity of a fabric care product comprising perfume microcapsules with the specific container 2, array 7 of sheets 6, and fabric care product comprising perfume microcapsules, as claimed herein. Without wishing to be bound by theory, it is believed that when removing the sheets 6 from the container 2 through the opening 3, friction occurs between the opening 3 and the sheets 6 that are removed from the opening 3. An amount of perfume microcapsules are ruptured by such friction, releasing perfume contained in the capsules, which in turn causes a burst of perfume. Preferably, as the sheets 6 are removed from the container 2, the arrangement of the sheets 6 enables friction to occur between sheets 6 that are contacted with one another within the container 2. This enhances the burst of perfume. In this way, an ordinary user is able to assess the fragrance intensity of a fabric care product comprising perfume microcapsules efficiently, without requiring complicated procedures or instruments.

Upon removal of each sheet 6 from the container 2 through the opening 3, the construction of the opening 3 provides for a similar or even the same amount of friction to be generated between the opening 3 and the sheet 6, thus maintaining steady assessing conditions.

Moreover, the preferred design of the edges 4 with protrusions 5 is such that as one sheet 6 is removed from the container 2 through the opening 3, it enables more friction to occur between the opening 3 and the sheet 6 as claimed herein. The above preferred edges cause stronger friction, thus breaking an even larger amount of perfume microcapsules.

In terms of the array 7 of sheets 6, at least 33% of the surface area of each sheet 6 in the array 7 of sheet 6 is in contact with one or more sheets 6 forming part of the array 7, and thus when a sheet 6 is removed from the container 2, a friction is generated between sheets 6 that are contacted one another within the container 2. Moreover, the preferred array 7 of sheets 6, i.e., a stack of sheet 6 wherein each sheet 6 overlaps with the neighboring sheet 6 over at least 50% of the surface area of one side of said sheet 6, enables a stronger friction between sheets 6, thus breaking a larger amount of perfume microcapsules.

More preferably, the arrangement of the sheets 6 is such that as the sheets 6 are removed from the container 2, it enables feeding of the next neighboring sheet 6 automatically. This automatic feeding of the next neighboring sheet 6 enables the kit 1 to work instantly for each assessment and to work for multiple times without intermittence.

### Method for Assessing the Fragrance Intensity

The present invention further encompasses a method for assessing the fragrance intensity imparted by a fabric care product comprising perfume microcapsules comprises step a) of providing the kit 1, and step b) of removing the sheets 6 from the container 2 through the opening 3.

Preferably, step a) comprises pulling the sheet 6 out of the container 2 through the opening 3, preferably by holding one end of a sheet partially expanding out of the container or reaching through the opening into the container. The benefits as described herein above for the kit 1, also apply to the method of the present invention.

In one embodiment of the present invention, after step b), the method further comprises a step c) of rubbing the sheets 6. Step c) breaks more perfume microcapsules and thus causes a stronger burst of perfume.

On one hand, the method assesses the fragrance intensity of the fabric care product comprising perfume microcapsules, and on the other hand, demonstrates the benefits of perfume microcapsules in general, i.e., without or small perfume leakage until being activated and causing a burst of perfume upon rupturing. Furthermore, the method allows users to have not only a delightful scent experience, but also an in-depth impression about perfume microcapsules. Specifically, by the above method, users will be left a good idea of how their fabrics will smell when they use the fabric care products comprising perfume microcapsules.

### Preparation

The process for preparing the kit 1 of the present invention comprises the step of making a container 2 with an opening 3; applying a fabric care product comprising perfume microcapsules onto sheets 6; arranging the sheets 6 to an array 7; and disposing the array 7 of sheets 6 into the container 2.

When the fabric care product comprising perfume microcapsules is granular or powder-form, a suitable method for applying the fabric care product onto sheets 6 may include gluing the granular or powder-form fabric care product onto the sheets 6. In another embodiment, the suitable method for applying the fabric care product onto sheets 6 may include dissolving the granular or powder-form fabric care product in aqueous solution, impregnating the sheets 6 within the aqueous solution comprising perfume microcapsules, and preferably drying.

When the fabric care product comprising perfume microcapsules is an aqueous solution, a suitable method for applying the fabric care product onto sheets 6 may include impregnating the sheets 6 within the fabric care product comprising perfume microcapsules, and preferably drying.

### Technical Data

For the following Examples, a Baseline is taken as the reference for generating the fragrance intensity data. The Baseline represents sheets with a perfume microcapsule-containing fabric care composition applied thereon having an ability to release a baseline fragrance intensity. PI I and PI II represent different embodiments of the present invention. PI I and PI II use the same sheets as compared to the Baseline. Thus, the fragrance intensity released by sheets that are disposed in the container of the kit of the present invention (PI I and PI II) is compared to the fragrance intensity released by the sheets that are not disposed in the container of the kit of the present invention (Baseline).

To assess the fragrance intensity, expert panelists smell Baseline, PI I, and PI II and assess the fragrance intensity, respectively. Each sheet is evaluated 9 times by 3 different panelists and each panelist repeats the evaluation 3 times.

For Baseline, panelists smell the sheets without pulling or rubbing. For PI and PI II, panelists pull sheets from a container according to the present invention and smell the sheets.

The fragrance intensity score is evaluated at a 0-100 scale. A difference of more than 5 grades versus Baseline represents a significant difference that is consumer noticeable. The grading scale is as follows:

100=extremely strong perfume odor

90=very strong-extremely strong

80=very strong

70=strong

60=moderate-strong

50=moderate

40=moderate

30=slight-moderate

20=slight

10=very slight

0=no perfume odor

### EXAMPLES

### Example 1

### Formulations of Fabric Care Products Comprising Perfume Microcapsules

The following compositions are made comprising the listed ingredients in the listed proportions (weight %). Composition A represents a composition of a fabric softening product comprising perfume microcapsules, and composition B represents a composition of a laundry detergent product comprising perfume microcapsules. The Examples herein are meant to exemplify the present invention but are not used to limit or otherwise define the scope of the present invention.

| | EXAMPLES | |
|---|---|---|
| (% wt) | A | B |
| FSA a | 12.1% | — |
| Surfactant system | — | |
| Sodium linear C11-13 alkyl benzene | | 8.8% |
| sulfonate | | |
| C12-14 alcohol ethoxylate AE7 | | 0.4% |
| C12-14 alcohol ethoxylate AE7 | | 6.0% |
| C24E3S b | | 2.0% |
| Citric acid | — | 1.6% |
| C12-18 Fatty acid | — | 4.0% |
| Lupamin ® 1595 c | 0.01% | — |
| Rheovis ® CDE d | 0.23% | — |
| Chelant e | — | 0.08% |
| Brightener f | — | 0.06% |
| Boric acid (MEA borate) | — | 1.48% |
| NaOH | — | 2.03% |
| NaCS | — | 0.40% |
| Ethanolamine | — | 0.82% |
| Ethanol | — | 1.43% |
| 1,2 propanediol | — | 1.52% |
| Dye | 0.00105% | 0.001% |
| Perfume Microcapsule (% active) | 0.25% | 0.2% |
| Perfume | 0.9% | 0.4% |
| Water | Add to 100% | Add to 100% |

a Ethanaminium, 2-hydroxy-N-(2-hydroxyethyl)-N,N-dimethyl, esters with C16-18 and C18-unsaturated Fatty acids, chlorides.
b C12-C14 alkyl sulfate condensed with 3 moles of ethylene oxide.
c Stabilizer available from BASF.
d Rheology modifier available from BASF.
e Phosphonic acid, [[(phosphonomethyl)imino]bis[2,1-ethanediylnitrilobis(methylene)]]tetrakis-, sodium salt
f Benzenesulfonic acid, 2,2′-(1,2-ethenediyl)bis[5-[[4,6-bis(phenylamino)-1,3,5-triazin-2-yl]amino]-, sodium salt (1:2)

### Example 2

### Process for Preparing the Kit Comprising a Fabric Softening Product Applied Under Machine Washing Conditions

The process for preparing the kit comprising a fabric softening product applied under machine washing conditions comprises:

a) preparing a plastic container with a rectangular opening having two parallel long edges and two parallel short edges, the edges having triangle-shaped protrusions extending from the edge portion of the opening into the opening portion, protrusion density being 12.5 protrusions per 1 cm length and protrusion depth being 1 mm, and the material forming the edge portion of the opening being dual layer of metal plate and cartoon;

b) preparing sheets of 100% cotton muslin, each sheet having a size of 20 cm*20 cm;

c) preparing a fabric softening product of composition A;

d) cleaning a washing machine thoroughly, adding 30 L of water into the washing machine, adding 30 g of liquid detergent of Pigeon Act'z® into the water, agitating 2 minutes to dissolve the detergent, putting 20 pieces of sheets into the water making sure all the sheets immerse in the water, washing for 17 minutes, and draining the water and spinning for 2 minutes;

e) adding 30 L of water into the washing machine, rinsing for 3 minutes, draining the water and spinning for 2 minutes, adding 30 L of water into the washing machine, rinsing for 3 minutes, and draining the water and spinning for 2 minutes;

f) adding 30 L of water into the washing machine, adding 67 g of the fabric softening product of composition A into the water, agitating 30 seconds to dissolve, putting the load into the water, agitating for 3 minutes, soaking for 7 minutes, and spinning for 2 minutes;

g) indoor line drying for 12 hours;

h) folding the sheets into a stack of 20 sheets, each sheet overlapping with the neighboring sheet at 50% of the surface area of one side of the sheet;

i) disposing the stack of sheets into the container.

### Example 3

### Process for Preparing the Kit Comprising a Fabric Softening Product Applied Under Machine Washing Conditions

The process for preparing the kit comprising a fabric softening product applied under machine washing conditions is the same as in Example 2, except for the following: a) the opening is rectangular having two parallel long edges and two parallel short edges without protrusions, the length of short edges being 0.1 mm, and the material of the opening being cartoon.

### Example 4

### Process for Preparing the Kit Comprising a Fabric Softening Product Applied Under Hand Washing Conditions

The process for preparing the kit comprising a fabric softening product applied under hand washing conditions comprises:

a) preparing a plastic container with a rectangular opening having two parallel long edges and two parallel short edges, the edges having triangle-shaped protrusions extending from the edge portion of the opening into the opening portion, protrusion density being 12.5 protrusions per 1 cm length and protrusion depth being 1 mm, and the material forming the edge portion of the opening being dual layer of metal plate and cartoon;

b) preparing sheets of 100% cotton muslin, each sheet having a size of 20 cm*20 cm;

c) preparing a fabric softening product of composition A;

d) cleaning a basin thoroughly, adding 10 L of water into the basin, adding 11.15 g of the fabric softening product of composition A into the water, agitating 30 seconds to dissolve, putting 20 pieces of sheets into the solution, agitating for 3 minutes, soaking for 7 minutes, and spinning for 2 minutes;

e) indoor line drying for 12 hours;

f) folding the sheets into a stack of 20 sheets, each sheet overlapping with the neighboring sheet at 50% of the surface area of one side of the sheet;

g) disposing the stack of sheets into the container.

### Example 5

### Process for Preparing the Kit Comprising a Fabric Softening Product Applied Under Hand Washing Conditions

The process for preparing the kit comprising a fabric softening product applied under hand washing conditions is the same as in Example 4, except for the following: a) the opening is rectangular having two parallel long edges and two parallel short edges without protrusions, the length of short edges being 0.1 mm, and the material of the opening being cartoon.

### Example 6

### Process for Preparing the Kit Comprising a Laundry Detergent Product Applied Under Machine Washing Conditions

The process for preparing the kit comprising a laundry detergent product applied under machine washing conditions comprises:

a) preparing a plastic container with a rectangular opening having two parallel long edges and two parallel short edges, the edges having triangle-shaped protrusions extending from the edge portion of the opening into the opening portion, protrusion density being 12.5 protrusions per 1 cm length and protrusion depth being 1 mm, and the material forming the edge portion of the opening being dual layer of metal plate and cartoon;

b) preparing sheets of 100% cotton muslin, each sheet having a size of 20 cm*20 cm;

c) preparing a liquid laundry detergent product of composition B;

d) cleaning a washing machine thoroughly, adding 30 L of water into the washing machine, adding 75 g of composition B into the water, agitating 2 minutes to dissolve composition B, putting 20 pieces of sheets into the water making sure all the sheets immerse in the water, washing for 17 minutes, and draining the water and spinning for 2 minutes;

e) adding 30 L of water into the washing machine, rinsing for 3 minutes, draining the water and spinning for 2 minutes, adding 30 L of water into the washing machine, rinsing for 3 minutes, and draining the water and spinning for 2 minutes, adding 30 L of water into the washing machine, rinsing for 3 minutes, draining the water and spinning for 2 minutes;

f) indoor line drying for 12 hours;

g) folding the sheets into a stack of 20 sheets, each sheet overlapping with the neighboring sheet at 50% of the surface area of one side of the sheet;

h) disposing the stack of sheets into the container.

### Example 7

### Comparative Data

A comparative experiment of assessing the fragrance intensity is conducted according to the Technical Data Section as described herein above. For the machine washing conditions, Baseline represents sheets produced by steps b) to e) of Example 2, PI I is the kit of Example 3, and PI II is the kit of Example 2. Likewise, for the hand washing conditions, Baseline represents sheets produced by steps b) to e) of Example 4, PI I is the kit of Example 5, and PI II is the kit of Example 4.

The below table shows the experimental results:

| Conditions | Panelist | Baseline | PI I | PI II |
|---|---|---|---|---|
| Machine | Panelist 1 | 30 | 35 | 35 |
| Washing | | 30 | 35 | 35 |
| | | 30 | 35 | 35 |
| | Panelist 2 | 30 | 35 | 35 |
| | | 30 | 35 | 35 |
| | | 30 | 35 | 35 |
| | Panelist 3 | 30 | 40 | 40 |
| | | 30 | 40 | 40 |
| | | 30 | 40 | 40 |
| Hand | Panelist 1 | 15 | 20 | 25 |
| Washing | | 15 | 20 | 25 |
| | | 15 | 20 | 25 |
| | Panelist 2 | 15 | 20 | 20 |
| | | 15 | 20 | 20 |
| | | 15 | 20 | 25 |
| | Panelist 3 | 15 | 20 | 20 |
| | | 15 | 20 | 20 |
| | | 15 | 20 | 20 |

The Kits according to the present invention, either PI I or PI II, show higher fragrance intensity by as much as 5 to 10 grades differences versus Baseline. As indicated in the Technical Data Section, a difference of more than 5 grades versus Baseline represents a significant difference that is consumer noticeable.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A kit for assessing the fragrance intensity of a fabric care product, said kit (1) comprising:
a) a container (2) comprising an opening (3);
b) an array (7) of sheets (6) disposed within said container (2), comprising at least two sheets (6), wherein at least 33% of the surface area of each sheet (6) in said array (7) is in contact with one or more sheets (6) forming part of said array (7); and
c) said fabric care product comprising perfume microcapsules wherein said fabric care product is applied onto said sheets (6),
wherein said sheets (6) have an ability to release a baseline fragrance intensity and are individually removed from said container (2) through said opening (3) by a user and wherein friction caused by such removal causes a fragrance intensity from said sheets (6) which is at least about 5 grades greater than said baseline fragrance intensity.

2. The kit according to claim 1, wherein the friction occurs between said opening (3) and said sheets (6) that are removed from said container (2) through said opening (3).

3. The kit according to any of the preceding claims, wherein the friction further occurs between said sheets (6) that are contacted one another within said container (2).

4. The kit according to any of the preceding claims, wherein said array (7) of sheets (6) is a stack of sheets (6).

5. The kit according to any of the preceding claims, wherein each sheet (6) overlaps with the neighboring sheet (6) over at least 50% of the surface area of one side of said sheet (6).

6. The kit according to any of the preceding claims, wherein said fabric care product is an aqueous solution, and is impregnated into said sheets (6) and dried within said sheets (6) prior to said sheets (6) being disposed within said container (2).

7. The kit according to any of the preceding claims, wherein said perfume microcapsules in said fabric care product for preparing said sheets (6) is able to deliver a solution with a concentration of perfume microcapsules from 0.1 ppm to 200 ppm, preferably from 0.2 ppm to 100 ppm, more preferably from 0.3 ppm to 50 ppm.

8. The kit according to any of the preceding claims, wherein the shape of said opening (3) is rectangular, oval, or round.

9. The kit according to any of the preceding claims, wherein said opening (3) is a rectangle having two parallel long edges and two parallel short edges, wherein the length of said short edges is from 0.05 mm to 40 mm, preferably from 0.1 mm to 5 mm, more preferably from 0.1 mm to 1.5 mm, and the length of said long edges is from 5 mm to 500 mm, preferably from 10 mm to 200 mm, more preferably from 15 mm to 150 mm.

10. The kit according to any of the preceding claims, wherein said opening (3) has one or more edges (4) with protrusions (5) extending from the edge portion of the opening (3) into the opening portion, wherein the shape of said protrusion (5) is selected from the group consisting of triangle, pentagon, hexagon, oval, and round, and a combination thereof.

11. The kit according to claim 10, wherein protrusion density along any one of the edges (4) is from 0.5 to 200, preferably from 0.25 to 37.5, more preferably from 0.125 to 25 protrusions per 1 cm length.

12. The kit according to any of claims 10 and 11, wherein protrusion depth along any one of the edges (4) is from 0.05 mm to 20 mm, preferably from 0.1 mm to 5 mm, more preferably from 0.1 mm to 1.5 mm.

13. The kit according to any of the preceding claims, wherein the material forming the edge portion of said opening (3) is selected from the group consisting of plastic, paper, cartoon, and metal plate, and a combination thereof in preferably two or more layers.

14. The kit according to any of the preceding claims, wherein the material of said sheets (6) is selected from the group consisting of cotton, poly cotton, nylon, paper, and nonwoven, and a combination thereof.

15. The kit according to any of the preceding claims, wherein said sheets (6) have applied thereon, preferably are printed with, pictures, product logos, or text.

16. The kit according to any of the preceding claims, wherein said fabric care product further comprises a perfume ingredient.

17. A method for assessing the fragrance intensity imparted by a fabric care product comprising perfume microcapsules, said method comprising
a) providing a kit (1) according to any of the preceding claims;
b) removing said sheets (6) from said container (2) through said opening (3).

18. The method according to claim 17, wherein after step b) said method further comprises:
c) rubbing said sheets (6).
